(19) United States
(12) Patent Application Publication
(10) Pub. No.: US 2023/0174915 A1
(43) Pub. Date: Jun. 8, 2023

(54) AEROBIC FERMENTATION SYSTEMS AND METHODS OF USING THE SAME

(71) Applicant: ARBELA LABORATORIES, INC., Randolph, NJ (US)

(72) Inventor: Michael LOCASCIO, Randolph, NJ (US)

(21) Appl. No.: 17/924,589

(22) PCT Filed: May 20, 2021
(86) PCT No.: PCT/US2021/033479

§ 371 Date: Nov. 10, 2022

Related U.S. Application Data
(60) Provisional application No. 63/028,167, filed on May 21, 2020.

(51) Int. Cl. C12M 1/00

(52) U.S. Cl. CPC C12M 29/18; C12M 47/02

(57) ABSTRACT

The present disclosure relates to an integrated methanol synthesis and fermentation system for the production of whole ells and biomolecules, and methods of using the same. In one embodiment, an apparatus comprises an inlet port; a pump in fluid communication with the inlet port to pump in a fermentation broth from a fermentation vessel; a cooling system; an aeration system in fluid communication with the cooling system; and an outlet port to reintroduce the fermentation broth into the fermentation vessel.

### CROSS-REFERENCE TO RELATED APPLICATION(S)

This application claims the benefit of priority of U.S. Provisional Patent Application No. 63/028,167, filed May 21, 2020, the disclosure of which is hereby incorporated by reference herein in its entirety.

### TECHNICAL FIELD

The present disclosure is directed to biomolecule production and, more specifically, to systems and methods of producing biomolecules, such as proteins, and single cell protein (whole cells) via fermentation processes.

### BACKGROUND

Biomolecules are typically large complex molecules produced by living organisms that include, but are not limited to peptides, proteins, enzymes, fatty acids, carotenoids, flavonoids, carbohydrates, and biopolymers (e.g., polyhydoxyalkanoates including polyhydroxybutyrate, chitin, cellulose, and pullulan). Biomolecular synthesis via fermentation is a well-established process that utilizes numerous types of single cell organisms ranging from bacteria, yeasts, mammalian cells, and algae typically, but not exclusively, grown in closed vessels under strict temperature conditions, aerobic or anaerobic conditions, and other conditions. In addition to complex biomolecules, simpler molecules, including low molecular weight alcohols, acids, and ketones, are commonly produced via fermentation.

Technological advancements over the past several decades have allowed for genetic engineering of many organism types that direct them to produce selected molecules. Alternatively, unaltered cells also naturally produce a variety of biomolecules and are often grown as sources of bulk protein or enzymes. Biomolecules including, but not limited to, proteins and other molecules described above, can either be excreted into a fermentation medium in which the single cell organisms are growing or are alternatively retained within the cell. In the former situation, the biomolecule can be separate from the fermentation medium using techniques including, but not limited to, ultrafiltration, precipitation, centrifugation, and high-performance liquid chromatography (HPLC). In the latter situation, the desired biomolecules may either be retained within whole cells (which are typically dried) or separated via cell lysing/rupturing or other well-known separation and purification processes. Applications of proteins produced via fermentation processes include biologic pharmaceuticals, analytic proteins, industrial enzymes, and bulk protein for human and animal nutrition (referred to as “single cell protein,” or “SCP”). Other fermentation applications include, but are not limited to, the production of nutritional supplements, biopolymers used in packaging and medical applications.

### SUMMARY

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its purpose is to present some concepts of the disclosure in a simplified form as a prelude to the more detailed description that is presented later.

Aspects of the present disclosure relate to a system and method for improving temperature control and dissolved oxygen levels of large-scale aerobic fermentation systems used to produce whole cell products or biomolecules.

In one aspect, a fermentation system comprises: a fermentation vessel; and an external loop in fluid communication with the fermentation vessel. In at least one embodiment, the external loops comprises: one or more inlet ports; one or more pumps in fluid communication with the one or more inlet ports to pump in a fermentation broth from the fermentation vessel; one or more outlet ports to reintroduce the fermentation broth into the fermentation vessel; a cooling apparatus; and an aeration apparatus in fluid communication with the cooling apparatus.

In at least one embodiment, the aeration apparatus is upstream from the cooling apparatus and the pump. In at least one embodiment, the cooling apparatus is upstream from the aeration apparatus and the pump.

In at least one embodiment, the aeration apparatus is configured to introduce an oxygen-containing gas into the fermentation broth. In at least one embodiment, the oxygen-containing gas comprises purified oxygen, air, or mixtures of oxygen with other gases.

In at least one embodiment, the aeration apparatus comprises one or more of a jet aerator, a surface aerator, or a fine bubble diffuser.

In at least one embodiment, the aeration apparatus comprises a nanobubble generator configured to produce bubbles of oxygen having a median diameter of less than about 200 nanometers.

In at least one embodiment, an inlet of the cooling apparatus is in fluid communication with an outlet of the pump. In at least one embodiment, the cooling apparatus comprises: one or more tubes through which the fermentation broth can flow; and a heat exchanger in thermal communication with the one or more tubes.

In at least one embodiment, the heat exchanger comprises one or more heat pipes. In at least one embodiment, a proximal end of at least one heat pipe is in thermal communication with the one or more tubes. In at least one embodiment, a distal end of the at least one heat pipe is in thermal communication with a coolant.

In at least one embodiment, the heat exchanger comprises or more of a shell and tube heat exchanger, a counterflow heat exchanger, a parallel flow heat exchanger, a plate heat exchanger, a plate-fin heat exchanger, a phase-change heat exchanger, or a microchannel heat exchangers. In at least one embodiment, the heat exchanger is configured to flow a coolant through a jacket in thermal communication with the one or more tubes.

In at least one embodiment, the coolant comprises one or more of air, chilled water, or a refrigerant. In at least one embodiment, the coolant is further in thermal communication with a chiller to maintain a temperature of the coolant below a temperature of the fermentation broth.

In at least one embodiment, the chiller comprises an adsorption chiller.

In at least one embodiment, the cooling apparatus further comprises a temperature sensor on an inlet side and/or an outlet side of the cooling apparatus.

In at least one embodiment, the fermentation system further comprises a media metering apparatus for introduction of media into fermentation broth. In at least one embodiment, the media comprises one or more of methanol, methane, glucose, dextrose, ethanol, sugar, glycerol, or yeast extract. In at least one embodiment, the media comprises methanol.

In at least one embodiment, the fermentation system further comprises one or more methanol sensors.

In at least one embodiment, the fermentation system further comprises one or more dissolved oxygen sensors.

In at least one embodiment, the fermentation system further comprises a separation apparatus for continuous separation of a portion of whole cells present in the fermentation broth. In at least one embodiment, an inlet of the separation apparatus is in fluid communication with the fermentation broth and at least two outlet streams. In at least one embodiment, a first outlet stream provides at least a portion of the whole cell depleted stream back into the apparatus and a second outlet stream provides a whole cell concentrated stream that is removed from the apparatus.

In at least one embodiment, the fermentation system further comprises a separation apparatus for the separation of a portion of a cleaned fermentation broth present in the fermentation broth, the cleaned fermentation broth containing biomolecules produced by cells in the fermentation broth. In at least one embodiment, an inlet of the separation apparatus is in fluid communication with the fermentation broth and at least two outlet streams. In at least one embodiment, a first outlet stream provides a cleaned fermentation broth depleted stream back into the apparatus and a second outlet stream provides a cleaned fermentation broth concentrated stream that is removed from the apparatus.

In at least one embodiment, the separation apparatus comprises one or more of a precipitator, a microfilter, an ultrafilter, a nanofilter, a crossflow filter, a centrifuge, or a continuous flow centrifuge.

In at least one embodiment, the fermentation system further comprises a CO₂ removal apparatus. In at least one embodiment, the CO₂ removal apparatus is configured to extract a portion of dissolved CO₂ from the fermentation broth.

In at least one embodiment, the CO₂ removal apparatus comprises a gas exchange membrane.

In at least one embodiment, the cooling apparatus is configured to maintain a temperature of the fermentation broth between 20° C. and 40° C.

In at least one embodiment, the aeration apparatus is configured maintain a dissolved oxygen level of the fermentation broth above 15%.

In at least one embodiment, the one or more inlet ports are located above the one or more outlet ports. In at least one embodiment, the one or more outlet ports are in fluid communication with a bottom portion of the fermentation vessel.

In at least one embodiment, one or more of the outlet ports are fitted with a diffuser.

In another aspect, a fermentation system comprises: a fermentation vessel; and at least one external loop in fluid communication with the fermentation vessel. In at least one embodiment, the external loops comprises: a cooling apparatus; and an aeration apparatus in fluid communication with the cooling apparatus. In at least one embodiment, the cooling apparatus and the aeration apparatus are disposed within different external loops.

In another aspect, a method of aerobic fermentation comprising utilizing a fermentation system of any of the aforementioned embodiments to produce whole cell products and/or biomolecules. In at least one embodiment, the biomolecules include proteins, enzymes, carotenoids, vitamins, biopolymers, lipids, cellulose, other molecules produced via fermentation processes, or combinations thereof. In at least one embodiment, the aerobic fermentation comprises growth of methylotrophic organisms. In at least one embodiment, the methylotrophic organisms comprise a yeast. In at least one embodiment, the yeast comprises Pichia pastoris. In at least one embodiment, the aerobic fermentation comprises growth of bacteria. In at least one embodiment, the bacteria comprises one or more of Methyophilus methylotrophus, Methylobacterium extorquens, Methylomonas methanolica, or Pseudomonas methanolica. In at least one embodiment, the biomolecules are excreted and separated from the fermentation broth or retained within the whole cell.

In another aspect, a method comprises: receiving a fermentation broth from a fermentation vessel into inlet ports of one or more external loops; causing the fermentation broth to flow through a cooling apparatus; causing the fermentation broth to flow through an aeration apparatus; and causing the fermentation broth to exit the apparatus and be reintroduced into the fermentation vessel via one or more outlet ports.

In at least one embodiment, the fermentation broth is flowed through the cooling apparatus prior to the aeration apparatus. In at least one embodiment, the fermentation broth is flowed through the aeration apparatus prior to the cooling apparatus.

In at least one embodiment, the aeration apparatus introduces an oxygen-containing gas into the fermentation broth. In at least one embodiment, the oxygen-containing gas comprises purified oxygen, air, or mixtures of oxygen with other gases.

In at least one embodiment, the aeration apparatus comprises one or more of a jet aerator, a surface aerator, or a fine bubble diffuser.

In at least one embodiment, the aeration apparatus comprises a nanobubble generator configured to produce bubbles of oxygen in the fermentation broth having a median diameter of less than about 200 nanometers.

In at least one embodiment, the cooling apparatus comprises: one or more tubes through which the fermentation broth flows; and a heat exchanger in thermal communication with the one or more tubes.

In at least one embodiment, the heat exchanger comprises one or more heat pipes. In at least one embodiment, a proximal end of at least one heat pipe is in thermal communication with the one or more tubes. In at least one embodiment, a distal end of the at least one heat pipe is in thermal communication with a coolant.

In at least one embodiment, the heat exchanger comprises or more of a shell and tube heat exchanger, a counterflow heat exchanger, a parallel flow heat exchanger, a plate heat exchanger, a plate-fin heat exchanger, a phase-change heat exchanger, or a microchannel heat exchangers. In at least one embodiment, the heat exchanger flows a coolant through a jacket in thermal communication with the one or more tubes.

In at least one embodiment, the coolant comprises one or more of air, chilled water, or a refrigerant. In at least one embodiment, the coolant is further in thermal communication with a chiller to maintain a temperature of the coolant below a temperature of the fermentation broth.

In at least one embodiment, the chiller comprises an adsorption chiller.

In at least one embodiment, the cooling apparatus further comprises a temperature sensor on an inlet side and/or an outlet side of the cooling apparatus.

In at least one embodiment, the method further comprises introducing media into the fermentation broth via a media metering apparatus. In at least one embodiment, the media comprises one or more of methanol, methane, glucose, dextrose, ethanol, sugar, glycerol, or yeast extract. In at least one embodiment, the media comprises methanol.

In at least one embodiment, the method further comprises separating a portion of whole cells present in the fermentation broth into a whole cell depleted stream and a whole cell concentrated stream. In at least one embodiment, the whole cell depleted stream is provided back into the apparatus. In at least one embodiment, the whole cell concentrated stream is removed from the apparatus.

In at least one embodiment, the method further comprises separating a biomolecule-containing portion of a cleaned fermentation broth present in the fermentation broth into a cleaned fermentation broth depleted stream and a cleaned fermentation broth concentrated stream. In at least one embodiment, the cleaned fermentation broth depleted stream is provided back into the apparatus. In at least one embodiment, the cleaned fermentation broth concentrated stream is removed from the apparatus.

In at least one embodiment, the method further comprises extracting a portion of dissolved CO₂ from the fermentation broth.

In at least one embodiment, the cooling apparatus maintains a temperature of the fermentation broth between 20° C. and 40° C.

In at least one embodiment, the aeration apparatus maintains a dissolved oxygen level of the fermentation broth above 15%.

In another aspect, a method for the production of whole cell protein from methylotrophic organisms comprises: measuring wet cell weight (WCW) at successive time-points; determining a maximum rate of biomass growth measured as increase in mass of biomass within a fermentation vessel that accounts for any increase in fermentation broth volume within the fermentation vessel; and extracting whole cells at a rate equivalent to the maximum rate of biomass growth.

In at least one embodiment, the method further comprises determining the maximum rate of biomass growth by taking the time derivative of the product of WCW and biomass volume.

In at least one embodiment, the method further comprises adjusting a flow rate of fermentation broth exposed to a separation apparatus such that cell density corresponding to the maximum rate of biomass production is maintained.

In at least one embodiment, the method further comprises lysing a portion of the extracted whole cells. In at least one embodiment, a product of the lysed whole cells is introduced into growth media. In at least one embodiment, the growth media is introduced into the fermentation broth or is used in a different fermentation process. In at least one embodiment, the methylotrophic organisms of comprise Pichia pastoris. In at least one embodiment, the methylotrophic organisms comprise one or more of Methyophilus methylotrophus, Methylobacterium extorquens, Methylomonas methanolica, or Pseudomonas methanolica.

In another aspect, any embodiments of the foregoing fermentation systems may be adapted to perform any embodiments of the foregoing methods.

In another aspect, any embodiments of the foregoing systems may comprise any embodiments of the foregoing separation apparatuses.

As used herein, the singular forms “a,” “an,” and “the” include plural references unless the context clearly indicates otherwise. Thus, for example, reference to “a protein” can include a single protein, multiple proteins of a single type, and mixtures of two or more different proteins.

Also as used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In at least one embodiment, the term “about” includes the recited number±1%, such that “about 10” would include 9.9 to 10.1 and all values in between.

Also as used herein, “protein” has its ordinary and customary meaning in the art and includes, and refers to a polypeptide (i.e., a string of at least two amino acids linked to one another by peptide bonds). Polypeptides may include natural amino acids, non-natural amino acids, synthetic amino acids, amino acid analogs, and combinations thereof. The term “peptide” is typically used to refer to a polypeptide having a length of less than about 50 amino acids. Proteins may include moieties other than amino acids (e.g., glycoproteins) and may be processed or modified. A protein can be a complete polypeptide chain as produced by a cell, or can be a functional portion thereof. A protein can include more than one polypeptide chain which may be chemically linked (e.g., by a disulfide bond), non-chemically linked (e.g., by hydrogen bonding), or both. Polypeptides may contain L-amino acids, D-amino acids, or both, and may contain any of a variety of amino acid modifications or analogs known in the art.

### BRIEF DESCRIPTION OF THE DRAWINGS

The present disclosure is illustrated by way of example, and not by way of limitation, in the figures of the accompanying drawings, in which:

FIG. 1 is a plot illustrating heat evolution and oxygen demand based on carbon source.

FIG. 2A is a block diagram illustrating an exemplary fermentation system for growing whole cells and/or synthesis of biomolecules in accordance with at least one embodiment of the disclosure.

FIG. 2B is a block diagram illustrating an exemplary fermentation system that includes multiple external loops in accordance with at least one embodiment of the disclosure.

FIG. 3A is a block diagram illustrating components of an exemplary external loop in accordance with at least one embodiment of the disclosure;

FIG. 3B is a block diagram illustrating components of a further exemplary external loop in accordance with at least one embodiment of the disclosure; and

FIG. 4 is a flow diagram illustrating a method of producing biomolecules or whole cells in accordance with at least one embodiment of the disclosure.

### DETAILED DESCRIPTION

Single cell protein (SCP) is an established technology already used for animal and human consumption albeit at far smaller scale than traditional sources of protein. SCP fermented on a methanol substrate using high protein content (60-80%) methylotrophic microorganisms offers a potential solution to produce protein needed for a growing population while greatly reducing the agricultural footprint. Protein can also be provided through the cultivation of various microbes and algae, preferably those which contain more than 30% protein in their biomass and which can provide a healthy balance of essential amino acids. Microbial protein is generally referred to as SCP, although some of the producing microbes, such as filamentous fungi or filamentous algae, may be multicellular. Sugar, sugar derivatives, or glycerol are often used as carbon and energy sources for growing cells (with additional nitrogen, salts, and other nutrient additives). Some organisms can utilize other molecules as carbon and energy sources. For example, methylotrophs are microorganisms that can utilize methanol or other simple alcohols as carbon and energy sources.

In addition to direct use as SCP, microbes contribute to protein demand when they are used to upgrade the protein content or quality of fermented foods. Although, microbial protein provides a relatively small proportion of current human nutrition, the growing global demand for protein is likely to make SCP increasingly important. High growth rates or the ability to utilize unique substrates, such as CO₂, methane, or methanol, result in processes which offer much higher efficiency and/or sustainability than is possible from traditional agriculture.

SCP is currently produced from a limited number of microbial species, although the range of sources for SCP used in animal feed is broader than that approved for human consumption and is expanding. Products derived from algae, fungi (including yeast) and bacteria are all in use or under development. The production steps generally include (a) preparation of nutrient media, (b) cultivation, including solid state fermentation, (c) separation and concentration of SCP, and in some cases drying, and (d) final processing of SCP into ingredients and products. SCP for human consumption is generally produced from food grade substrates and regulatory issues must always be considered.

A wide range of fungi have been considered for use as SCP. Products from Saccharomyces, Fusarium, and Torulopsis are commercially available. Fungi grown as SCP will generally contain 30-50% protein. Methylotrophic yeasts, for example Komagataella pastoris (previously Pichia pastoris), produce biomass and protein from methanol. Bacteria also have a long history of use as SCP, particularly in animal feed. Bacterial SCP generally contains 50-80% protein on a dry weight basis. As with fungi, bacterial SCP has high nucleic acid content (8-12%), in particular RNA, and thus requires processing prior to usage as food/feed. In addition to protein, bacterial SCP provides some lipid and B group vitamins.

All fermentation applications are very sensitive to cost and full market adoption is contingent upon reducing costs below alternatives. Large-scale bulk protein applications for human and animal nutrition are particularly cost sensitive as they compete with cheap soymeal, the largest source of plant-based protein and the primary component in animal feed.

Presently, the most common method of maintaining a fermentation vessel at a temperature for optimal microbial growth (approximately 28° C.) is through the use of a cooling jacket in connection with all or a portion of the exterior surface of a fermentation vessel in which a coolant (typically chilled water) flows. However, the surface area to volume ratio of a fermentation vessel decreased as the size of the vessel increases given as the volumes increase with r³ (where r is the approximate radius of the vessel) while surface area increases with r². As such, the heat flow needed to maintain a given temperature must increase accordingly and becomes a practical engineering limitation.

Similarly, it is a challenge to maintain adequate dissolved oxygen levels as oxygen demand (i.e., the amount of oxygen used by the microorganisms) increases at elevated cell growth rates and cell densities. It is furthermore a challenge to maintain uniform oxygenation throughout the fermentation vessel as volume increases. Conventional aeration techniques include bubbling air or purified oxygen though the fermentation broth. However, conventional aeration has relatively poor oxygen transfer rates owing to the size of the bubbles and because most of the bubbles reach the upper surface and exit the broth without complete transfer of oxygen. Bubbling itself becomes more challenging and energy intensive at elevated viscosities observed at high cell concentrations.

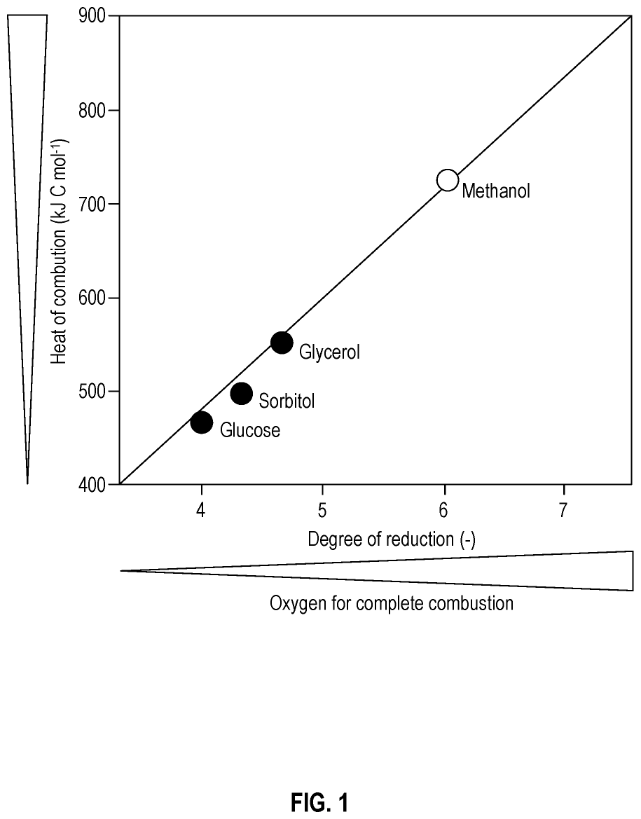

The thermal control and oxygenation issues described above are particularly problematic for fermentations using methanol fed Pichia pastoris. Pichia pastoris metabolizes methanol for both energy and biomass accumulation using alcohol oxidase enzymes (AOX) that reacts dissolved oxygen with methanol to form formaldehyde that, in turn, is the substrate for downstream metabolic reactions. It is the alcohol oxidation that is both the source for elevated oxygen consumption and heat generation (see FIG. 1).

These and other limitations of current systems are addressed by the present disclosure, which describes apparatuses, systems incorporating apparatuses, and methods to improve the temperature control and dissolved oxygen levels of large-scale aerobic fermentation systems used to produce whole cell products or biomolecules. Nonlimiting examples of biomolecules include proteins, enzymes, carotenoids, vitamins, biopolymers, lipids, cellulose, other molecules produced via fermentation processes, and combinations thereof. Aerobic fermentation includes, but is not limited to, the growth of methylotrophic organisms, with nonlimiting examples of each including yeasts such as Pichia pastoris as well as bacteria such as Methyophilus methylotrophus, Methylobacterium extorquens, Methylomonas methanolica, Pseudomonas methanolica, and others. In at least one embodiment, biomolecules may either be excreted and separated from a fermentation broth or retained within the whole cell. Whole cells containing biomolecules can then either separated and dried or alternatively extracted via cell separation, lysing, and biomolecule purification. With respect to protein, applications include the production of heterologous protein/peptide biopharmaceuticals, industrial enzymes, and analytical proteins, as well as bulk protein that may either be extracted or retained within whole cells and used as human and animal nutritional additives.

As used herein, “fermentation broth” refers to an aqueous solution/suspension comprising whole cells, water, media, biomolecules excreted by the whole cells, and other constituents within the fermentation vessel. The broth can be separated into the whole cell fraction and a cleaned fermentation broth. The cleaned fermentation broth comprises water, media, biomolecules excreted by the cells, and other constituents.

Also as used herein, “media” refers to a solution comprising the carbon and chemical energy source (typically one or more of glucose, sugars, methanol, glycerol, or other carbon containing molecules), water, and optionally one or more of nitrogen containing molecules (e.g., ammonia salts), phosphate, and other salts and nutrients, including optional yeast extract, needed to promote microbial growth.

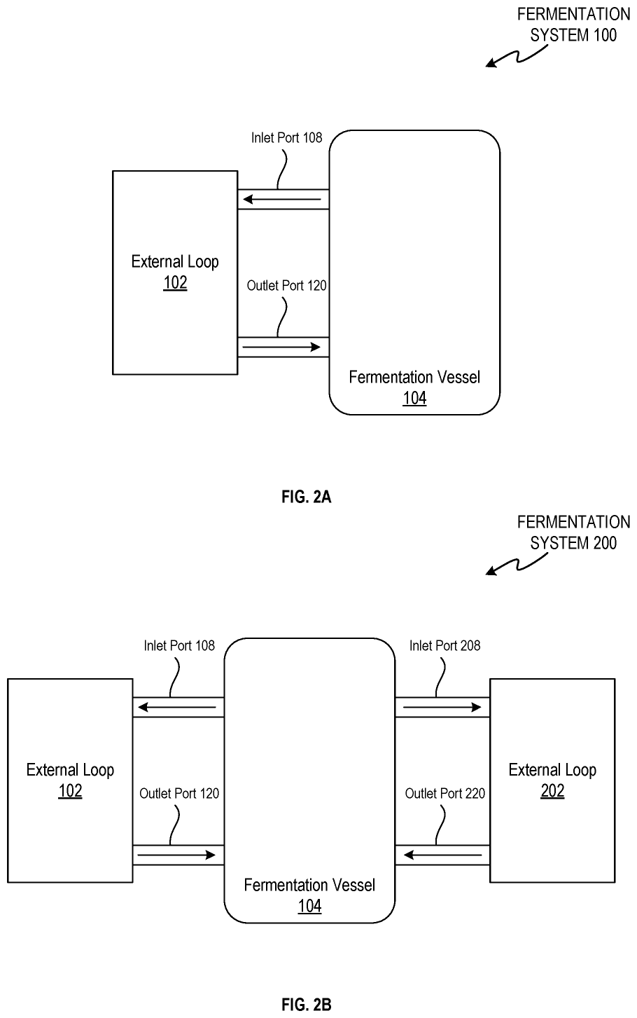

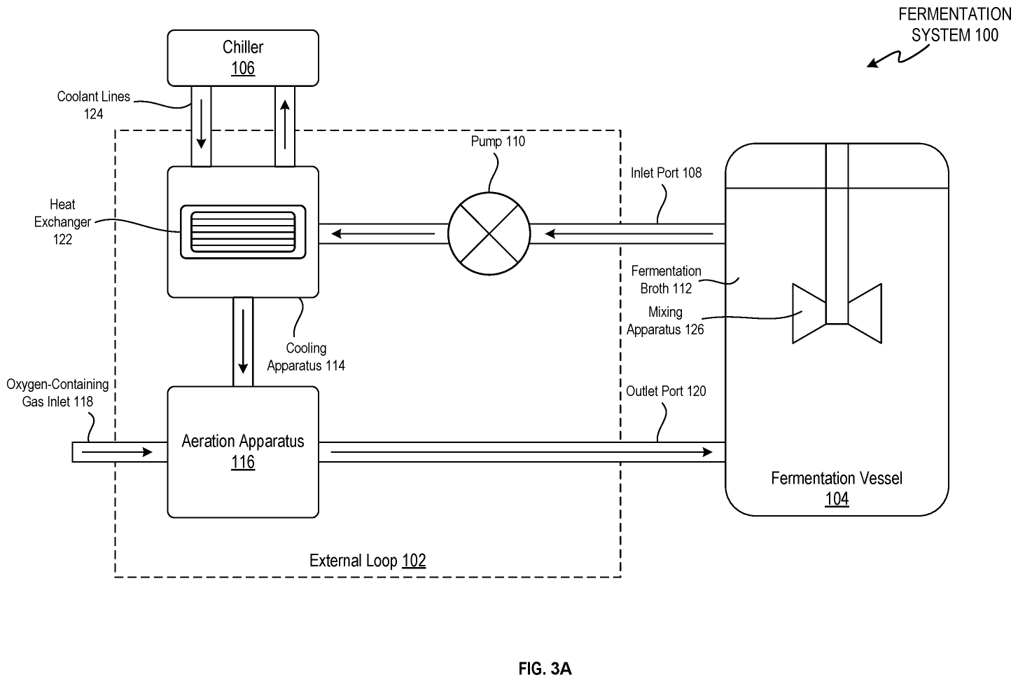

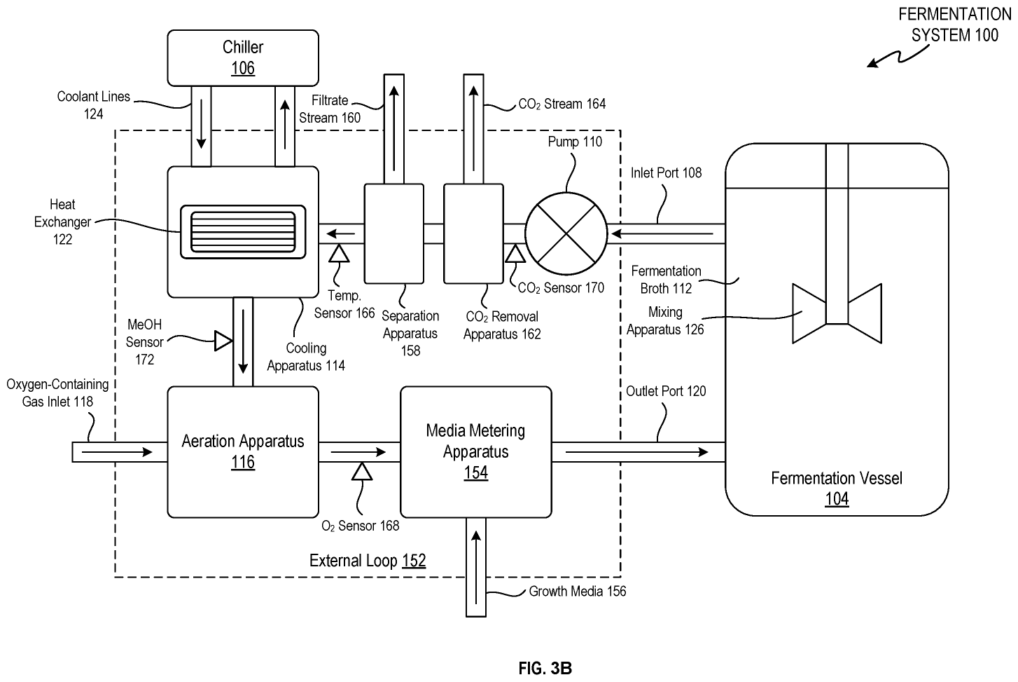

FIG. 2A is a block diagram illustrating an exemplary fermentation system 100 for growing whole cells and/or synthesis of biomolecules in accordance with at least one embodiment of the disclosure. The fermentation system 100 comprises an external loop 102 for treating that is in fluid communication with a fermentation vessel 104 via an inlet port 108 and an outlet port 120. The external loop 102 comprises various components for treating a fermentation broth received from the inlet port 108 (e.g., near an upper portion of the fermentation vessel 104) and reintroducing the treated fermentation broth back into the fermentation vessel 104 via the outlet port 120 (e.g., near a lower portion of the fermentation vessel 104). The components of the external loop 102 are described in greater detail below with respect to FIGS. 3A and 3B.

FIG. 2B is a block diagram illustrating an exemplary fermentation system 200 that includes multiple external loops in accordance with at least one embodiment of the disclosure. For example, as illustrated, the fermentation system 200 comprises the external loop 102 as well as an external loop 202 (which may be the same or similar to the external loop 102) that is in fluid communication with the fermentation vessel 104 via an inlet port 208 and an outlet port 220. It is to be understood that the use of two external loops is exemplary, and that additional external loops may be present. It is to be further understood that the fermentation systems 100 and 200 are not drawn to scale, may designed to be of any suitable dimensions, and may be modified as desired as would be appreciated by those of ordinary skill in the art. It is to be understood that one or more of the components of fermentation systems 100 and 200 can be optional. In at least one embodiment, some or all of the functionality of each of fermentation systems 100 or 200 may be automated.

FIG. 3A is a block diagram illustrating the components of the external loop 102 in accordance with at least one embodiment of the disclosure. In addition to the external loop 102 and the fermentation vessel 104, the fermentation system 100 further includes a chiller 106 in fluid communication with the external loop 102.

In at least one embodiment, the external loop 102 includes the inlet port 108 in fluid communication with the fermentation vessel 104, one or more pumps 110 that pushes higher temperature deoxygenated fermentation broth 112 in a circuit through the external loop 102, a cooling apparatus 114 that removes heat from the fermentation broth 112, an aeration apparatus 116 that dissolves oxygen from an oxygen-containing gas inlet 118 into the fermentation broth 112, and the outlet port 120 that returns cooled and oxygenated fermentation broth 112 to the fermentation vessel 104. In at least one embodiment, the cooling apparatus 114 precedes the aeration apparatus 116 because oxygen dissolves more readily in cooled aqueous solutions including the fermentation broth 112 than in warmer aqueous solutions. In at least one other embodiment, the aeration apparatus 116 precedes the cooling apparatus 114. In at least another embodiment, the outlet port 120 is fitted with a diffuser, which facilitates the mixing of the fermentation broth. In a preferred embodiment, the inlet port 108 (and additional inlet ports, if present) are located above the outlet port 120 (and additional outlet ports, if present) such that fermentation broth 112 is reintroduced into a lower portion or the bottom of the fermentation vessel 104.

In at least one embodiment, the fermentation vessel 104 may further be equipped with a mixing apparatus 126, defoaming mechanisms, an array of sensors (sensors for measuring, for example, temperature, turbidity, oxygenation, CO₂, MeOH, etc.), or other components as would be appreciated by those of ordinary skill in the art.

In at least one embodiment, the pump 110 is in fluid communication with the inlet port 108, though it is to be understood that other pumps may be placed in the circuit (e.g., after the cooling apparatus 114 or the aeration apparatus 116) to facilitate maintaining an appropriate flow rate.

In at least one embodiment, the cooling apparatus 114 includes a heat exchanger 122 from which thermal energy in the fermentation broth 112 is transferred to a coolant circulated through the chiller 106 via coolant lines 124. Various technologies can be modified for use in with the present embodiments including, but not limited to, a tube-in-shell design for which the fermentation broth 112 flows through one or more tubes within a jacket in which a coolant flows. In at least one embodiment, the coolant may be chilled water, air, or a refrigerant. In at least one embodiment, the coolant may flow in the same direction or in a countercurrent manner. Other exemplary heat exchangers that can be modified for use with the present embodiments include fin or plate designs in which heat conducting metals are thermally coupled to a conducting tub or tubes through which the fermentation broth 112 flows. Heat can flow from the fermentation broth 112 to the fins or plates that are in turn cooled by air flow, chilled water, or a refrigerant. In all cases, the heat exchanger may be designed such that the coolant remains in the same phase or alternatively undergoes a phase change (e.g., evaporation).

In at least one embodiment, heat pipes may be used to efficiently transfer heat from the proximal end to the distal end. In such embodiments, the proximal end is placed in thermal communication with the fermentation broth 112 while the distal end is in thermal communication with a coolant. Heat pipes are generally metal tubes in which there is a working fluid and a wick. Heat at the proximal end causes the working fluid absorb heat and evaporate, which then travels to the proximal end where is condenses and releases heat into the coolant. The condensed working fluid is then wicked back to the proximal end of the heat pipe via capillary action. In at least one embodiment, the chiller 106 may include one or more heat pipes that extend into the fermentation vessel, and may include one or more mixing impellers including, but not limited to, Rushton impellers, paddle mixers, and helical mixers.

In at least one embodiment, heat is removed from the coolant and released into the environment by the chiller 106. In at least one embodiment, the chiller may include, but is not limited to, adsorption chillers that are powered by heat energy including steam, solar energy, or combustion of natural gas, oil, or other fuels.

In at least one embodiment, a temperature of the fermentation broth 112 is maintained between 10° C. and 50° C., 20° C. and 30° C., 25° C. and 35° C., or 26° C. and 29° C.

In at least one embodiment, the aeration apparatus 116 serves to dissolve oxygen into the fermentation broth 112. The oxygen may be received from an oxygen-containing gas inlet 118 that provides air, purified oxygen, or mixtures of oxygen gas with other gases, such as CO₂, argon, N₂, or other gaseous species (such as volatile organic compounds). Purified oxygen may be optionally generated using techniques known in the art including, but not limited to, temperature swing adsorption and pressure swing adsorption systems that utilize adsorbents, such as molecular sieves, zeolites, and other materials.

In at least one embodiment, the oxygen-containing gas may be introduced into the fermentation broth 112 via a variety of methods including, but not limited to, bubbling, jet aeration, sparging, spraying the fermentation broth 112 through oxygen-containing gases, or other methods known in the art. In at least one embodiment, oxygen is introduced into the fermentation broth 112 by the generation of nanoscale bubbles (e.g., less than 200 nanometers), which are small enough to have a surface charge that helps maintains them in suspension. Nanobubbles have been demonstrated to vastly increase oxygen transfer by at least a factor of three over conventional methods in the art (see, for example, U.S. Patent Application Publication Nos. 2016/0236158 A1 and 2014/0191425 A1).

In at least one embodiment, a dissolved oxygen level of the fermentation broth 112 is maintained above 5%, above 10%, above 15%, above 20%, above 25%, or above 30%.

In at least one embodiment, when multiple external loops are utilized (e.g., as in the fermentation system 200), one or more components illustrated in FIG. 3A may be separated across the multiple external loops. For example, in at least one embodiment, the aeration apparatus 116 may be disposed in a different external loop than the heat exchanger 122 (e.g., the aeration apparatus 116 may be disposed within the external loop 102 while the heat exchanger 122 may be disposed within the external loop 202). In such embodiments, the locations of the heat exchanger 122 and the aeration apparatus 116 can vary provided that the fermentation system 100 or 200 includes at least one heat exchanger 122 and at least one aeration apparatus 116. In at least one embodiment, multiple aeration apparatuses 116 and heat exchangers 122 may be present.

FIG. 3B is a block diagram illustrating an external loop 152 that is a modified version of the external loop 102 in accordance with at least one embodiment of the disclosure. The external loop 152 further includes: a media metering apparatus 154 for the introduction of growth media 156 into the fermentation broth 112; a separation apparatus 158 for removing a portion of the whole cells from the fermentation broth 112 as a filtrate stream 160 or a portion of cleaned fermentation broth (including biomolecules suspended in cleaned fermentation broth) as the filtrate stream 160; a CO₂ removal apparatus 162 for extracting CO₂ from the fermentation broth 112 into a CO₂ stream 164; one or more temperature sensors 166; one or more oxygen sensors 168; one or more CO₂ sensors 170; and one or more methanol sensors 172 (which may be present if methanol is included in the growth media 130). In at least one embodiment, exemplary methanol sensors 172 may be provided by Raven Biotech or Sartorious Stedim Biotech. In at least one embodiment, exemplary oxygen sensors 168 may be provided by Hach, Sensorex, Hamilton, or Process Instruments. In at least one embodiment, exemplary CO₂ sensors 170 may be provided by Mettler Toledo, Anton-Paar, or Martek Instruments.

In at least one embodiment, the media metering apparatus 154 is configured to introduce the growth media 156 into the fermentation broth 112. Nonlimiting examples of growth media 156 may include a carbon and energy source, salts (including nitrate and/or phosphate salts), biotin, yeast extracts, or other components. Carbon and energy sources may include, but are not limited to, glucose, dextrose, other sugars, glycerol, methane, methanol, ethanol, and combinations thereof.

An exemplary growth medium for Pichia pastoris is now described. The growth medium (referred to as buffered glycerol-complex medium (BMGY) or buffered methanol-complex medium (BMMY) depending on the carbon source used) includes 1% yeast extract, 2% peptone, 100 mM potassium phosphate (pH 6.0), 1.34% yeast nitrogen base (with ammonium sulfate). BMGY further includes 0.75% glycerol, while BMMY further includes 1% methanol.

A further exemplary growth medium is now described, which is a mixture of FM22 powder and PMT1 salts. FM22 powder (Sunrise Science Products, Cat #4090) contains: potassium phosphate, monobasic (42.9 g/L), ammonium sulfate (5 g/L), calcium sulfate dehydrate (1 g/L), potassium sulfate (14.3 g/L), and magnesium sulfate anhydrous (5.71 g/L). For high cell density fermentations, the FM22 powder can be used at 68.9 g/L. PTM1 salts contain: biotin (0.2 g/L), boric acid (0.02 g/L), cobalt chloride anhydrous (equivalent to 0.28 g/L), copper sulfate.5H₂O (6 g/L), iron sulfate anhydrous (35.51 g/L), magnesium sulfate.H₂O (3 g/L), sodium iodide (0.08 g/L), sodium molybdate.2H₂O (0.2 g/L), and zinc chloride (20 g/L). The PTM1 salts can be solubilized at 65.3 g/L with 5 mL of H₂SO₄/liter, with 4 mL/liter of the mixture being used for high cell density fermentation.

In at least one embodiment, the separation apparatus 158 removes all or a portion of whole cells from the fermentation broth into a whole cell concentrated filtrate stream 160 while returning the remainder into the fermentation broth via a second whole cell depleted stream. Nonlimiting examples of the separation apparatus 158 include precipitation, microfiltration, ultrafiltration, nanofiltration, centrifugation, constant flow centrifugation, and other techniques known in the art.

Organisms dispersed within the fermentation broth 112 may or may not excrete protein or other biomolecules into the fermentation broth 112. All or a portion of the fermentation broth 112 with whole cells may be extracted from the fermentation vessel 104 into an output stream. In at least one embodiment, the output stream may be separated via a cell separation apparatus into a concentrated whole cell stream and a whole cell-depleted stream. In at least one embodiment, the whole cell-depleted stream may be recycled back into the fermentation vessel 104. In at least one embodiment, the concentrated whole cell stream may be subjected to lysis followed by purification to isolate the desired biomolecules which may introduced into a drying apparatus. In at least one embodiment, the concentrated whole cell stream (with protein, oils, and/or other biomolecules contained within the whole cells) is introduced into the drying apparatus to produce dried whole cells. Exemplary and non-limiting examples of a cell separation apparatus include centrifuges, continuous flow centrifuges, and filters, such as microfilters and ultrafilters having an average pore size smaller than the average diameters of the whole cells. In at least one embodiment, the drying apparatus may be effectuated directly via a superheated steam drying system, or indirectly by using steam heat to drive heated air dryers. In at least one embodiment, the heated air dryers may additionally incorporate a spray drying apparatus such that hydrated whole cells or excreted biomolecules are sprayed into droplets exposed to elevated temperature from superheated steam or dry air. Other non-limiting exemplary drying processes include freeze drying and lyophilization.

In at least one embodiment, a portion of whole cells separated into the filtrate stream 160 is between 0% and 1% of whole cell concentration measured as wet cell weight (WCW) within the fermentation broth 112. In at least one embodiment, the portion of whole cells separated into the filtrate stream 160 is between 0% and 10% of whole cell concentration measured as WCW within the fermentation broth 112. In at least one embodiment, the portion of whole cells separated into the filtrate stream 160 is between 0% and 25% of whole cell concentration measured as WCW within the fermentation broth 112. In at least one embodiment, the portion of whole cells separated into the filtrate stream 160 is between 0% and less than 50% of whole cell concentration measured as WCW within the fermentation broth 112. In at least one embodiment, the portion of whole cells separated into the filtrate stream 160 is between 0% and 99% of whole cell concentration measured as WCW within the fermentation broth 112.

When the product is in the form of biomolecules (e.g. proteins or other biomolecules) suspended in the cleaned fermentation broth, the separation apparatus 158 removes a portion of cleaned fermentation broth from the fermentation broth into a cleaned fermentation broth concentrated filtrate stream 160 while returning the remainder into the fermentation broth 112 via a second cleaned fermentation broth depleted stream. Nonlimiting examples include microfiltration, nanofiltration, centrifugation, constant flow centrifugation and other techniques known in the art.

In at least one embodiment, the fermentation system 100 further includes an excreted biomolecule separation apparatus that receives the filtrate stream 160 including the fermentation broth 112, whole cells, and biomolecules excreted from the whole cells. The excreted biomolecule separation apparatus may be a centrifuge, a crossflow filter, or other device capable of separating out higher molecular weight protein from the liquid fraction and smaller molecular weight fractions. In at least one embodiment, the excreted biomolecule separation apparatus may separate the output stream into a biomolecule-containing stream (which may be provided to a biomolecule purification apparatus) and a biomolecule-depleted stream (containing the fermentation broth 112 and whole cells) that is reintroduced into the fermentation vessel 104. In at least one embodiment, biomolecule purification apparatus may use one or more of the following techniques to obtain purified biomolecules including, but not limited to, ultrafiltration, nanofiltration, crossflow filtration, reverse osmosis, ultracentrifugation, precipitation, chromatography, and high pressure liquid chromatography.

In at least one embodiment, a portion of cleaned fermentation broth separated into the filtrate stream 160 is between 0% and 1% of cleaned fermentation broth concentration measured as (100%−WCW) within the fermentation broth 112. In at least one embodiment, a portion of cleaned fermentation broth separated into the filtrate stream 160 is between 0% and 10% of cleaned fermentation broth concentration measured as (100%−WCW) within the fermentation broth 112. In at least one embodiment, a portion of cleaned fermentation broth separated into the filtrate stream 160 is between 0% and 25% of cleaned fermentation broth concentration measured as (100%−WCW) within the fermentation broth 112. In at least one embodiment, a portion of cleaned fermentation broth separated into the filtrate stream 160 is between 0% and 50% of cleaned fermentation broth concentration measured as (100%−WCW) within the fermentation broth 112. In at least one embodiment, a portion of cleaned fermentation broth separated into the filtrate stream 160 is between 0% and 99% of cleaned fermentation broth concentration measured as (100%−WCW) within the fermentation broth 112.

In at least one embodiment, the CO₂ removal apparatus 162 is configured to remove CO₂ derived from the aerobic respiration of the microorganism from the fermentation broth 112. For example, the CO₂ removal apparatus 162 may utilize polyamines or CO₂-selective membranes. CO₂ removal apparatuses that can be modified for use with the present embodiments are described, for example, in U.S. Pat. Nos. 5,271,743 and 8,647,569. In at least one embodiment, selective CO₂ membranes can include one or more hollow gas permeable fibers optionally coated with siloxane, and may optionally be rotated within the fermentation broth 112 to reduce the gas boundary layer and facilitate gas transfer into the hollow fiber.

The economic production of bulk single cell protein via requires both the maximum conversion of media feedstock to biomass to minimum operating costs (OPX), as well as the maximization of biomass growth rate. It should be noted that biomass growth involves not only increases in cell/biomass density (concentration) but also the increase in fermentation broth volume within the fermentation vessel as media is added and converted to biomass (e.g., fermentation broth volume within fermentation vessel increases over time). Total biomass (e.g., biomass density multiplied by volume) increases according to an S-shaped curve where it increases slowly at first at low cell densities, followed by a period of rapid expansion having a peak growth rate at moderate cell densities, followed by a phase of slower growth at high cell densities as the cells compete for oxygen and carbon molecules and other nutrients. As such, optimal economic production occurs at cell density corresponding to maximum growth. This can be determined by taking the derivative of biomass within the fermentation as a function of time where biomass at each time point is the WCW multiplied by the volume of fermentation within the fermentation vessel. WCW is measured using methods known in the art, with nonlimiting examples including measurement of optical density (OD) using the measurement of optical absorbance at 550 nanometers with a spectrophotometer.

In at least one embodiment, to maintain WCW within the fermentation vessel 104 at the cell density corresponding to maximum biomass increase, the separation apparatus 158 can be adjusted to remove whole cells at the same rate as the peak time derivative of the product of WCW and volume. Maximum heterologous protein (or other fermentation) production occurs at a different and usually much higher cell density. Nevertheless the same process as above can be applied with the exception that the separation apparatus 158 is set to remove whole cells at the time the derivative of the product of the WCW and volume (which is lower than the maximum biomass rate above) corresponds to the cell density at maximum heterologous or fermentation product rate.

Enriching growth media with yeast extract can accelerate growth rate. As such, the process may optionally include taking a portion of the whole yeast cells removed from the fermentation broth 112 via the separation apparatus 158, lysing the cells to form an extract, and introducing that extract into the growth media 156. It should be understood that yeast extract derived as explained above may be used within the same fermentation run from which it was obtained, or alternatively either used immediately in a different fermentation run or stored for later use in a different fermentation run.

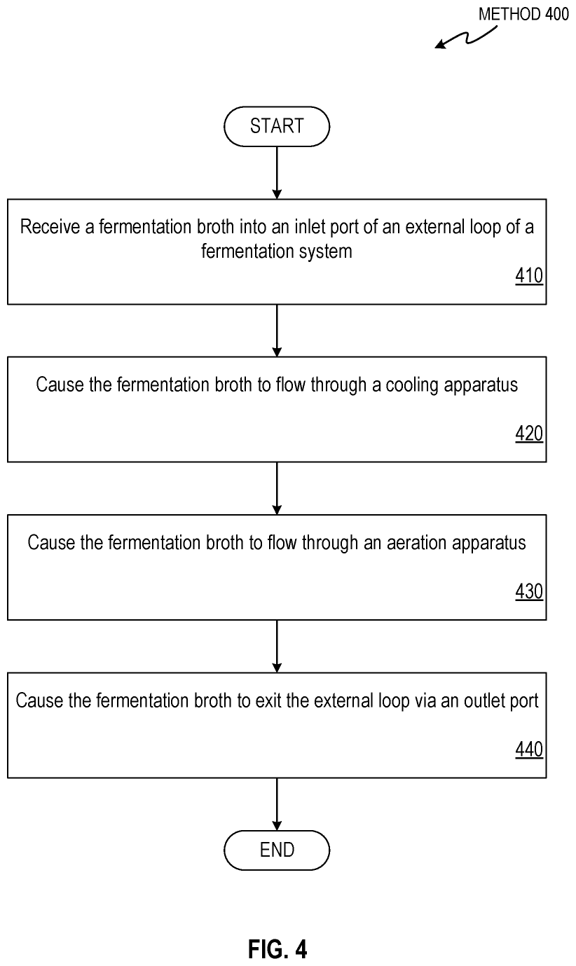

FIG. 4 is a flow diagram illustrating a method 400 of producing biomolecules or whole cells in accordance with at least one embodiment of the disclosure. The method 400 may be performed, in at least one embodiment, using the fermentation system 100 or the fermentation system 200.

At block 410, a fermentation broth (e.g., fermentation broth 112 from the fermentation vessel 104) is received into an inlet port of an apparatus (e.g., the inlet port 108 of the external loop 102 or 152, or the inlet port 208 of the external loop 202).

At block 420, the fermentation broth is flowed through a cooling apparatus (e.g., the cooling apparatus 114). In at least one embodiment, the flow of the fermentation broth is driven by a pump (e.g., the pump 110).

In at least one embodiment, the cooling apparatus comprises one or more tubes through which the fermentation broth flows, and a heat exchanger in thermal communication with the one or more tubes. In at least one embodiment, the heat exchanger comprises one or more heat pipes, where a proximal end of at least one heat pipe is in thermal communication with the one or more tubes. In at least one embodiment, a distal end of the at least one heat pipe is in thermal communication with a coolant. In at least one embodiment, the heat exchanger comprises or more of a shell and tube heat exchanger, a counterflow heat exchanger, a parallel flow heat exchanger, a plate heat exchanger, a plate-fin heat exchanger, a phase-change heat exchanger, or a microchannel heat exchangers.

In at least one embodiment, the heat exchanger flows a coolant through a jacket in thermal communication with the one or more tubes. In at least one embodiment, the coolant comprises one or more of air, chilled water, or a refrigerant. In at least one embodiment, the coolant is further in thermal communication with a chiller to maintain a temperature of the coolant below a temperature of the fermentation broth.

In at least one embodiment, the chiller comprises an adsorption chiller. In at least one embodiment, the cooling apparatus further comprises a temperature sensor on an inlet side and/or an outlet side of the cooling apparatus.

In at least one embodiment, the cooling apparatus maintains a temperature of the fermentation broth between 20° C. and 40° C.

At block 430, the fermentation broth is flowed through an aeration apparatus (e.g., the aeration apparatus 116). In at least one embodiment, the fermentation broth is flowed through the cooling apparatus prior to the aeration apparatus. In at least one embodiment, the fermentation broth is flowed through the aeration apparatus prior to the cooling apparatus.

In at least one embodiment, an oxygen-containing gas into the fermentation broth, which may comprise purified oxygen, air, or mixtures of oxygen with other gases. In at least one embodiment, the aeration apparatus comprises one or more of a jet aerator, a surface aerator, or a fine bubble diffuser. In at least one embodiment, the aeration apparatus comprises a nanobubble generator configured to produce bubbles of oxygen in the fermentation broth having a median diameter of less than about 200 nanometers.

In at least one embodiment, media is introduced into the fermentation broth via a media metering apparatus (e.g., the media metering apparatus 154). In at least one embodiment, the media comprises one or more of methanol, methane, glucose, dextrose, ethanol, sugar, glycerol, or yeast extract. In at least one embodiment, the media comprises methanol.

In at least one embodiment, the aeration apparatus maintains a dissolved oxygen level of the fermentation broth above 15%.

In at least one embodiment, the a portion of whole cells present in the fermentation broth is separated into a whole cell depleted stream and a whole cell concentrated stream. In at least one embodiment, the whole cell depleted stream is provided back into the apparatus, and the whole cell concentrated stream is removed from the apparatus.

In at least one embodiment, a biomolecule-containing portion of a cleaned fermentation broth present in the fermentation broth is separated into a cleaned fermentation broth depleted stream and a cleaned fermentation broth concentrated stream. In at least one embodiment, the cleaned fermentation broth depleted stream is provided back into the apparatus, and the cleaned fermentation broth concentrated stream is removed from the apparatus.

In at least one embodiment, a portion of dissolved CO₂ is extracted from the fermentation broth.

At block 440, the fermentation broth exits through an outlet port of the apparatus (e.g., the outlet port 120 of the external loop 102 or 152, or the outlet port 220 of the external loop 202) and is reintroduced into the fermentation vessel 104.

In the foregoing description, numerous specific details are set forth, such as specific materials, dimensions, processes parameters, etc., to provide a thorough understanding of the embodiments of the present disclosure. The particular features, structures, materials, or characteristics may be combined in any suitable manner in one or more embodiments. The words “example” or “exemplary” are used herein to mean serving as an example, instance, or illustration. Any aspect or design described herein as an “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the use of the terms “a,” “an,” “the,” and similar referents in the context of describing the materials and methods discussed herein (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Reference throughout this specification to “one embodiment,” “certain embodiments,” “one or more embodiments,” “an embodiment,” or “some embodiments” means that a particular feature, structure, material, or characteristic described in connection with the embodiment is included in at least one embodiment of the present disclosure. Thus, the appearances of the phrases such as “in one or more embodiments,” “in certain embodiments,” “in one embodiment,” or “in an embodiment” in various places throughout this specification are not necessarily referring to the same embodiment of the present disclosure. Furthermore, the particular features, structures, materials, or characteristics may be combined in any suitable manner in one or more embodiments.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

Although the embodiments disclosed herein have been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents, and the above-described embodiments are presented for the purposes of illustration and not of limitation.

1. A fermentation system comprising:
a fermentation vessel; and
an external loop in fluid communication with the fermentation vessel, wherein the external loops comprises:
one or more inlet ports;
one or more pumps in fluid communication with the one or more inlet ports to pump in a fermentation broth from the fermentation vessel;
one or more outlet ports to reintroduce the fermentation broth into the fermentation vessel;
a cooling apparatus; and
an aeration apparatus in fluid communication with the cooling apparatus.

2. The fermentation system of claim 1, wherein the aeration apparatus is upstream from the cooling apparatus and the pump.

3. The fermentation system of claim 1, wherein the cooling apparatus is upstream from the aeration apparatus and the pump.

4. The fermentation system of claim 1, wherein the aeration apparatus is configured to introduce an oxygen-containing gas into the fermentation broth, wherein the oxygen-containing gas comprises purified oxygen, air, or mixtures of oxygen with other gases.

5. The fermentation system of claim 1, wherein the aeration apparatus comprises one or more of a jet aerator, a surface aerator, or a fine bubble diffuser.

6. The fermentation system of claim 1, wherein the aeration apparatus comprises a nanobubble generator configured to produce bubbles of oxygen having a median diameter of less than about 200 nanometers.

7. The fermentation system of claim 1, wherein an inlet of the cooling apparatus is in fluid communication with an outlet of the pump, and wherein the cooling apparatus comprises:
one or more tubes through which the fermentation broth can flow; and
a heat exchanger in thermal communication with the one or more tubes.

8. The fermentation system of claim 7, wherein the heat exchanger comprises one or more heat pipes, wherein a proximal end of at least one heat pipe is in thermal communication with the one or more tubes, and wherein a distal end of the at least one heat pipe is in thermal communication with a coolant.

9. The fermentation system of claim 7, wherein the heat exchanger comprises or more of a shell and tube heat exchanger, a counterflow heat exchanger, a parallel flow heat exchanger, a plate heat exchanger, a plate-fin heat exchanger, a phase-change heat exchanger, or a microchannel heat exchangers, wherein the heat exchanger is configured to flow a coolant through a jacket in thermal communication with the one or more tubes.

10. The fermentation system of claim 8, wherein the coolant comprises one or more of air, chilled water, or a refrigerant, wherein the coolant is further in thermal communication with a chiller to maintain a temperature of the coolant below a temperature of the fermentation broth.

11-35. (canceled)

36. A method comprising:
receiving a fermentation broth from a fermentation vessel into inlet ports of one or more external loops;
causing the fermentation broth to flow through a cooling apparatus;
causing the fermentation broth to flow through an aeration apparatus; and
causing the fermentation broth to exit the apparatus and be reintroduced into the fermentation vessel via one or more outlet ports.

37. The method of claim 36, wherein the fermentation broth is flowed through the cooling apparatus prior to the aeration apparatus.

38. The method of claim 36, wherein the fermentation broth is flowed through the aeration apparatus prior to the cooling apparatus.

39. The method of claim 36, wherein the aeration apparatus introduces an oxygen-containing gas into the fermentation broth, wherein the oxygen-containing gas comprises purified oxygen, air, or mixtures of oxygen with other gases.

40. The method of claim 36, wherein the aeration apparatus comprises one or more of a jet aerator, a surface aerator, or a fine bubble diffuser.

41. The method of claim 36, wherein the aeration apparatus comprises a nanobubble generator configured to produce bubbles of oxygen in the fermentation broth having a median diameter of less than about 200 nanometers.

42. The method of claim 36, wherein the cooling apparatus comprises:
one or more tubes through which the fermentation broth flows; and
a heat exchanger in thermal communication with the one or more tubes.

43. The method of claim 42, wherein the heat exchanger comprises one or more heat pipes, wherein a proximal end of at least one heat pipe is in thermal communication with the one or more tubes, and wherein a distal end of the at least one heat pipe is in thermal communication with a coolant.

44. The method of claim 42, wherein the heat exchanger comprises or more of a shell and tube heat exchanger, a counterflow heat exchanger, a parallel flow heat exchanger, a plate heat exchanger, a plate-fin heat exchanger, a phase-change heat exchanger, or a microchannel heat exchangers, wherein the heat exchanger flows a coolant through a jacket in thermal communication with the one or more tubes.

45-55. (canceled)

56. A method for the production of whole cell protein from methylotrophic organisms, said method comprising:
measuring wet cell weight (WCW) at successive time-points;
determining a maximum rate of biomass growth measured as increase in mass of biomass within a fermentation vessel that accounts for any increase in fermentation broth volume within the fermentation vessel; and
extracting whole cells at a rate equivalent to the maximum rate of biomass growth.

57-63. (canceled)
